(19) United States
(12) Patent Application Publication
(10) Pub. No.: US 2007/0160680 A1
(43) Pub. Date: Jul. 12, 2007

(54) Fluid composition used to stimulate human synovial fluid

(75) Inventor: Thorsten Schwenke, Chicago, IL (US)

Correspondence Address: FOLEY & LARDNER LLP, 150 EAST GILMAN STREET, P.O. BOX 1497, MADISON, WI, 53701-1497, US

(73) Assignee: Rush University Medical Center

(21) Appl. No.: 10/588,680

(22) PCT Filed: Feb. 11, 2005
(86) PCT No.: PCT/US05/04551

§ 371 Date: Aug. 8, 2006

Related U.S. Application Data
(60) Provisional application No. 60/544,051, filed on Feb. 12, 2004.

(51) Int. Cl. A61K 35/16; A61K 31/7034; A61K 31/195

(52) U.S. Cl. 424/531; 514/35; 514/566

(57) ABSTRACT

An artificial synovial fluid comprising a serum, a chelator, and a buffer is provided. In particular embodiments, the artificial synovial fluid may further comprise an antibiotic. The artificial synovial fluid is generally used in tribological testing of medical device implants such as artificial joints. Methods of using and making the artificial synovial fluid are also provided.

### PRIORITY CLAIM

The present application specifically claims priority to U.S. Provisional Patent Application No. 60/544,051, filed Feb. 12, 2004. The entirety of this priority document is herein specifically incorporated by reference.

### FIELD OF THE INVENTION

The present invention relates to a fluid composition used to simulate synovial fluid in the tribological analysis of artificial joints. More specifically, the invention relates to a fluid composition used to simulate synovial fluid which generates wear properties similar to synovial fluid (involved in the tribological analysis of artificial joints).

### BACKGROUND OF THE INVENTION

In-vitro evaluation of implant performance is a standard practice in the design, development, and manufacture of artificial hip and knee joints. In order to obtain clinically relevant results from such tests, it is essential to simulate the in vivo joint conditions as closely as possible. Such joint conditions include the applied loads, moments and displacements, the temperature, and the surrounding media present in the joint.

Fluid compositions to be used during in vitro testing are commonly produced for the purpose of simulating the synovial fluid that naturally surrounds the joint in vivo. Various studies show the significant influence of chemical and physical parameters of testing fluid in the wear outcome in joint material testing. Nevertheless, the field of developing an artificial synovial fluid that approximates natural synovial fluid while generating clinically relevant results during implant testing is still in the infancy stages. The exact concentrations and types of components that will provide the best artificial synovial fluid have previously been unknown in the art. Therefore, a need exists to continue in the development of artificial synovial fluids.

### SUMMARY OF THE INVENTION

In one embodiment the present invention provides an artificial synovial fluid, comprising a serum, a chelating agent, and a buffer in an aqueous solution, wherein the artificial synovial fluid approximates natural synovial fluid and generates clinically relevant results during implant testing.

In many embodiments, the aqueous solution will be deionized water. In one aspect of the present invention, it is contemplated that the serum of the fluid composition is bovine calf serum. It is further contemplated that the chelator comprises Ethylene-Diamine-Tetra-Acetate (EDTA). The buffer of the invention may be phosphate buffered saline. The buffer may also be Tris-(hydroxymethyl)-aminomethane (tris). In certain embodiments, the serum, chelator, and buffer in aqueous solution will be in specific concentrations.

In another aspect, artificial synovial fluid will additionally have an antibiotic. It is contemplated that the antibiotic comprises sodium azide. It is additionally contemplated that the antibiotic may comprise Patricin.

The invention further contemplates the methods used to make the compositions of the artificial synovial fluid.

Additional aspects of the invention relate to the use of the artificial synovial fluid. Specifically, the artificial synovial fluid may be used in wear testing of implant medical devices.

### DETAILED DESCRIPTION OF THE INVENTION

Generally, the present invention encompasses an artificial synovial fluid composition comprising a protein source and a chelating agent in an aqueous solution. In certain embodiments, an antibiotic will be added to the artificial synovial fluid. Although not meant to be limiting, the artificial synovial fluid may be used in the tribological testing of implants. The implants tested using the artificial synovial fluid of the present invention may be implants designed for use in humans, nevertheless, the testing of veterinary implants will also benefit from the compositions of the present invention.

Any appropriate protein source may be used. In one aspect of the invention, the proteins may be derived through synthetic processes. In another embodiment, the protein source may be plasma, including blood plasma. In yet another embodiment, the protein source may be serum. The protein source also may be protein concentrate that was extracted from serum. Generally, serum encompasses any fluid component of blood derived or obtained from a living organism (in dried or liquid form), whether the organism is prenatal, postnatal, mature or adult. Examples of serum that may be used with the invention include, but are not limited to, bovine serum (such as bovine calf serum and fetal calf serum), ovine serum, canine serum, equine serum, caprine serum, human serum and porcine serum. The skilled artisan understands that substitution of the protein source, specifically substitution between different types of serum, may change the overall cost of production of the artificial synovial fluid. In a first embodiment of the present invention, when serum is used as the protein source, the serum is bovine calf serum.

Many protein sources, such as various types of serum, are commercially available. Purchased serum is advantageous because it generally has a specification sheet setting forth the protein concentration. However, individual measurements to measure the protein content of serum or any alternative protein source may be taken using testing methods known in the art. Because the protein concentration of the artificial synovial fluid is crucial, it is advantageous to determine the exact protein concentration of the serum. For examples of articles that discuss the importance of the protein concentration of artificial synovial fluids, see Saikko, J Tribology, 125, 638-642 (2003) and Bell et al. Proc Instn Mech. Engrs, Part H, J Eng Med, 214(H5): p. 513-8. (2000).

In some embodiments, it may be advantageous to use serum from a particular individual. In these embodiments, the protein concentration of the serum will need to be determined. If the artificial synovial fluid is used in vivo, in order to prevent rejection, serum can be isolated from the individual where the artificial synovial fluid will be used. In the cases where serum is isolated, the serum may be sterilized or purified before use. The skilled artisan understands that several well known methods exist for sterilizing and purifying serum.

The artificial synovial fluid also contains a chelating agent. Generally, any organic or inorganic compound that will bind to a metal ion having a valence greater than one may used in the invention as long as the purpose of the invention, which is to approximate natural synovial fluid while providing clinically relevant results during tribological implant testing, is maintained. Chelating agents include, but are not limited to, organic chelating agents such as EDTA, the sodium salts of EDTA, triethylene tetramine dihydrochloride (TRIEN), ethylene glycol-bis (β-aminoethyl ether-N,N,N′,N′-tetracetic acid (EGTA), diethylenetriamin-pentaacetic acid (DPTA), and triethylenetetramine hexaacetic acid (TTG), deferoxamine, Dimercaprol, edetate calcium disodium, zinc citrate, penicillamine succimer and Editronate or any other chelating agent that will chelate divalent ions such as Ca²⁺, Mg²⁺, Mn²⁺, Fe²⁺, and Zn²⁺, and which are acceptable for use with the present invention. In one embodiment, the chelating agent of the fluid composition comprises EDTA.

In some embodiments a single chelating agent will be used in the compositions, whereas in other embodiments, a mixture of chelating agents will be used. In certain embodiments, a particular chelating agent may be acceptable for one use of the artificial synovial fluid but not acceptable for a different use.

In certain embodiments, the concentration of the chelating agent may also be linked to the concentration of ions present in the serum. Linking the concentration of the chelating agent to the concentration of ions insures that excess chelating agent is not added to the fluid. This linking may both save on cost and prevent excess chelating agent from changing the properties of the artificial synovial fluid.

The aqueous solution of the invention may encompass pure deionized water, as well as saline or Ringers solution. In some embodiments, if water is used, the water need not be deionized water but can be distilled, filtered or treated with reverse osmosis. Nevertheless, in some embodiments where implants containing metal-metal combinations will be tested, the amount and type of ions in the water may need to be controlled for if the water being used is not deionized. This is especially true if the type of test being performed is an electro-conductivity test to see if the implant is subject to fretting corrosion. In deciding on the type of water to use with the invention, the skilled artisan understands that one of the goals of water treatment is to remove the biologics and additives that may be found in tap water.

In certain embodiments, the saline or Ringers solution will be purchased from a commercial supplier, such as Fisher Scientific, as a finished product. In other embodiments, the saline or Ringers solution may be custom mixed. An example of a non-limiting custom mixed saline solution includes adding 0.750 to 0.860 grams of NaCl, 0.021 to 0.033 grams of CaCl₂, and 0.030 to 0.035 grams of KCl to 100 ml of deionized water. The skilled artisan understands that this custom mixture is an example only and that custom and commercial saline mixtures having different components and concentrations may be used with the compositions of the invention.

In some embodiments, the aqueous solutions may be buffered. Any buffer that allows the artificial synovial fluid to approximate natural fluid while providing clinically relevant results may be used. As understood by the skilled artisan, a buffer maintains the stability of a solution. Because the typical tribological test lasts for 500 k cycles as 1 Hz cycle frequency (approximately 5.8 days at 37° C.), the more stable the fluid, the more likely that the fluid will maintain similar characteristics over the life of the test. In certain embodiments, the saline solution will be a phosphate buffered solution. To make a phosphate buffered solution, NaHPO₄ and KH₂PO₄ should be added to saline or Ringer's solution. The making of phosphate buffered solution is well within the purview of the skilled artisan and will not be discussed in further detail. As understood by the skilled artisan, a multitude of buffers may be used in a single embodiment of the artificial synovial fluid. In certain embodiments, an amount of a buffer such as Tris, may be used either alone or with the phosphate buffered solution.

In various embodiments of the invention, an antibiotic may be added to the artificial synovial fluid. As used herein, an antibiotic refers to an agent that has the ability to destroy or interfere with the development of living organisms. For use with the invention, antibiotics encompass fungicides and herbicides as well as anti-microbials. Because growth of fungi and microbes in the artificial synovial fluid may change the properties of the fluid, it is important that antibiotics be added to the compositions, especially if the artificial synovial fluid is stored.

In some embodiments, only a single antibiotic will be used in the artificial synovial fluid of the present invention. In other embodiments, a mix of antibiotics will be used. One of skill in the art will recognize that the type and amount of antibiotic is limited only in that the antibiotic must be capable of either controlling or preventing growth of biologics in the artificial synovial fluid without preventing the intended use of the artificial synovial fluid. Depending on the intended use of the artificial synovial fluid, the appropriate amount and type of antibiotic may change. Without undue experimentation, the skilled artisan can easily determine an appropriate antibiotic in an appropriate amount for use in the present invention.

An example of an appropriate antibiotic is Patricin. In some embodiments this antibiotic may be Patricin A. Although certain embodiments contain Patricin, many antibiotic candidates exist which can be freely substituted. For example, although certain embodiments use Patricin as the antibiotic, other antibiotics including Vemamycin, Virginiamycin or sodium azide may be used. Other applicable antibiotics include gentamicin and amphotericin.

Other additives to the artificial synovial fluid are contemplated. These additives may include substances such as hyaluronic acid and lipids such as dipalmitoyl phosphatidylcholine. For examples of additional synovial fluid additives, see U.S. Pat. No. 6,800,298 and U.S. Patent Application 2002/0143121. Because it is advantageous to keep the artificial synovial fluid stable as long as possible, additives that support the stability by preventing protein precipitation, bacterial growth, pH changes, and other stability defeating events may be used. Although, several of these additives include the disclosed buffers and antibiotics, the use of additional additives is anticipated.

In individual embodiments, the invention comprises about 25.0% to about 99.8% w/w bovine calf serum, which includes subranges of bovine calf serum such as 25% to 33%, 33% to 60%, and 60% to 99.8%, about 0.01% to about 3.0% w/w EDTA, which includes subranges of EDTA such as 0.01% to 0.1%, 0.1% to 0.74% and 0.74% to 3.0%, and up to about 67.0% w/w deionized water.

In another embodiment, sodium azide may be added to the above solution. When adding sodium azide, the manufacturer's recommendations should generally be followed. This results in a concentration about 0.1% to about 5.0% sodium azide w/w. Patricin A may also be added to the artificial synovial fluid, either alone or in combination with sodium azide. Similarly to the sodium azide, the final concentration of Patricin A should generally be based on the manufacturer's recommendations, resulting in a concentration of about 0.1% to about 5.0% Patricin A.

In the embodiments where Tris is used, the artificial synovial fluid may comprise about 25.0% to about 99.8% serum, about 0.01% to about 3.0% chelating agent, about 0.1% to about 5.0% antibiotic, about 1% to about 35% Tris, and aqueous solution up to about 67%. In certain embodiments using Tris, the artificial synovial fluid may comprise about 33.0% to about 60% serum, about 0.01% to about 0.74% chelating agent, 0.1% to about 5.0% antibiotic, about 1% to about 5% Tris, and aqueous solution up to about 67%. In some of the embodiments using Tris, antibiotic will not be added.

Although different embodiments may comprise different components and/or different ranges of components, when using a particular mix of components for tribological implant testing, it is important to be consistent in preparing the fluid for each use as batch to batch variability may impact test results. Commonly in these embodiments, approximately 20 to 30 grams of protein per liter of artificial synovial fluid will be used.

In making the artificial synovial fluid compositions of the present inventions, the components of the composition are mixed together. Although the components may be mixed in any order, the components should be thoroughly mixed. One way to ensure thorough mixing is to mix the components on a stir plate for a minimum of 15 minutes. Nevertheless, any type of mixing that results in thorough mixing but does not significantly denature the proteins in the artificial synovial fluid may be used.

In some embodiments, the serum will be warmed to 37° C. before addition of the other components. However, the skilled artisan understands that the temperature of the serum, as long as the serum is liquid and the temperature does not exceed the temperature where a significant amount of protein starts to denature, is not particularly critical. The serum used in the invention may either be fresh serum or serum that has been frozen and then thawed. Because the properties of artificial synovial fluids are believed to be controlled by the amount of protein denaturation in the composition, multiple freeze/thaw cycles of the serum are not recommended.

In many cases, the artificial synovial fluid will be sterile filtered before use, although this is not a requirement. If the artificial synovial fluid is to be sterile filtered, the filter should be chosen so that it removes a significant portion of the microbes and other biologics that may be present in the fluid. A common example of a filter that may be used for this purpose is a 0.22 micron filter. The sterilization filters can generally be of any material that does not interfere with the properties of the artificial synovial fluid. In some embodiments, prior to filter sterilization, the artificial synovial fluid may be filtered such as for clarifying purposes. Clarifying filtration is commonly done with a 0.45 micron filter. Once again, the clarifying filter may be of any material that does not interfere with the properties of the artificial synovial fluid.

In certain embodiments, the pH of the artificial synovial fluid may be changed by the addition of a base such as sodium hydroxide and/or the addition of an acid such as hydrochloric acid. In some embodiments, the pH of the artificial synovial fluid will be adjusted until the pH approximates physiological pH (commonly 7.4 in humans). As stated above, the artificial synovial fluid may contain a buffer which stably maintains the pH of the fluid. The pH of the artificial synovial fluid may be adjusted at any time. As a non-limiting example, the pH may be adjusted after the fluid has been mixed. The pH may also be adjusted after the fluid has been stored.

Once the artificial synovial fluid has been prepared, it can be stored for a limited amount of time. A non-limiting example of storage conditions include refrigerating the fluid at −20° C. for 10 or fewer days. However, if the artificial synovial fluid takes on a contaminated appearance, it should be discarded.

In the embodiments where the artificial synovial fluid is used in tribological testing of artificial joints, the fluid is typically filled in a fully enclosed chamber that contains the joint being tested. Enough fluid is placed in the fully enclosed chamber so that the contact surfaces of the artificial joint are submersed. In certain embodiments, the artificial synovial fluid may be direct injected into an area containing a contact surface. When the artificial synovial fluid is used to test artificial joints, the fluid will commonly be replaced at regular intervals. This interval may be daily, every other day, every third day, or beyond every third day. In one embodiment, instead of replacing the artificial synovial fluid, the fluid will be continuously refreshed. Continual refreshment prevents the fluid from becoming contaminated and also prevents the proteins in the fluid from becoming denatured.

The medical implants that may be tested with the fluid are not particularly limiting and may include any artificial bone implant such as implants used in fracture fixation such as pedicle screws or artificial joints such as hip, knee, spine, shoulder, and elbow joints. Generally, the implants fall into two classes. The first class is those implants that are implanted in the body that need to withstand the harsh in vivo conditions of the body with respect to electro-chemical/biological resistance, and mechanical as aspects such as fatigue and micro-motion. The second class is artificial joints that undergo, in addition to the previous challenges, larger motions that create a higher amount of wear. The skilled artisan understands that the type of implant generally determines the type of testing.

In the case of fracture fixation implants, the tribological testing will be primarily fatigue testing, a method of testing well known to the skilled artisan. Concerning artificial joints, wear testing as well as fatigue testing may be carried out in the artificial synovial fluid of the present invention. Although implant testing may be done at any time, implant testing is typically done throughout the development phase of a product implant. For example, the Food and Drug Administration (FDA) requires that all new implants be tested prior to FDA approval. In certain cases, implant testing may be done while a particular type of implant is already in use in order to provide experimental evidence of implant performance. For an exemplary example of how to test the wear of a device similar to a total hip prostheses, see Clark, Wear 250: 188-198 (2001).

Generally, the specific artificial synovial fluid used to test implants will depend on the composition of the implant. For example, the artificial synovial fluid that works best with the current Cobalt-Chromium, of which many implants are constructed, may not be optimal for implants made of different materials. The skilled artisan can easily determine the appropriate artificial synovial fluid for the particular implant material.

When measuring the specific properties of the artificial synovial fluid of the present invention, such as the impact the fluid has on implant wear, generally the density, viscosity, rheological behavior and conductivity of the fluid will be measured. In testing for the impact of the artificial synovial fluid on specific wear properties, wear tests with different embodiments of the fluid can be compared against each other and also against natural synovial fluid. The comparison generally includes comparing the amount and appearance of wear of the implants and also the characteristics of the wear particles in the fluid. Generally, the amount of wear of the implant will change with changing amounts of protein in the artificial synovial fluid. Other components of the artificial synovial fluid, including various additives such as chelators, may also change the wear characteristics. An artificial synovial fluid that approximates the natural synovial fluid and provides clinically relevant results consists of a fluid that results in similar wear patterns on the implant and/or similar morphology of generated particles during implant testing as compared to the wear patterns on the implant and the morphology of generated particles in an in vivo situation.

In some embodiments, the artificial synovial fluid will be used in testing implants following the current testing standards for medical devices. These testing standards include the testing standards developed by the International Standards Organization (ISO) and ASTM International Standards Worldwide. For an example of the types of testing standards currently in place for particular implants, please see ISO 14242-1 International Standard, 2002, Implants for Surgery—Wear of total hip-joint prosthesis—Part 1: Loading and displacement parameters for wear-testing machines with displacement control and corresponding environmental conditions for test, International Organization for Standardization, Geneva, Switzerland.

The present compositions are further illustrated by the following non-limiting examples.

### EXAMPLE 1

### Preparation of Artificial Synovial Fluid Used to Test an Artificial Hip

The following materials are used in preparing the artificial synovial fluid for testing an artificial hip: a) Newborn Calf Serum, b) Patricin A, c) EDTA, d) deionized water; e) mixing cylinder; f) heating bath capable of reaching 50° C.; g) magnetic stirrer and stir bar, h) filter unit 22 microns and i) filter unit 45 microns. Each of these components are available from multiple commercial suppliers.

In this example, the artificial synovial fluid is prepared as follows:

1. Pre-heat the frozen calf serum in water bath to 37-39° C.

2. Fill mixing cylinder with amount of calf serum needed for the target volume in an applicable amount

3. Add EDTA and Patricin A in an applicable amount

4. Fill up the cylinder to the desired fluid amount

5. Mix the fluid (magnetic stirrer) for at least 15 min

6. Filter the fluid first through the 0.45 micron filter, then through the 0.22 micron filter

7. Fill a container, such as a squeeze bottle, with the fluid for use in simulator chambers.

Approximately 300 ml of the fluid is then added to a Model HS2-12-1000, 12 Station Hip Simulator (AMTI-Boston) to test wear on artificial hips according to experimental protocols provided with the machine. Fluid is replaced in regular intervals of 1 to 3 days depending on the testing cycle.

### EXAMPLE 2

### Specific Examples of Synthetic Synovial Fluid Compositions

| | Final | | Deionized | | |
|---|---|---|---|---|---|
| | Vol | Serum | water | | Patricin |
| Composition | [ml] | [ml] | [ml] | EDTA [g] | [μg] |
| 1 | 1000 | 517.2 | 482.8 | 3.85 | 500 |
| | | (51.72%) | (48.28%) | (.385%) | (0.05%) |
| 2 | 1000 | 392.0 | 397.8 | | |
| | | (39.20%) | (39.78%) | | |
| | Final | | Phosphate | | |
| | Vol | Serum | Buffered | | |
| Composition | [ml] | [ml]* | Saline [ml] | EDTA [g] | Tris [g] |
| 3 | 1000 | 588.0 | 384.8 | 0.200 | 27.0 |
| | | (58.80%) | (38.48%) | (0.02%) | (2.7%) |
| 4 | 1000 | 392.0 | 397.8 | 5.8 | 0 |
| | | (39.20%) | (39.78%) | (0.58%) | |

*serum has a protein content of 51 g/l

As used herein a means “one” or “one or more.” As will be understood by one skilled in the art, for all purposes, particularly in terms of providing a written description, all ranges disclosed herein also encompass all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than,” and the like include the number recited and refer to ranges that can be subsequently broken down into subranges as discussed above. In the same manner, all ratios disclosed herein also include all subratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

All references disclosed herein are specifically incorporated by reference thereto.

While preferred embodiments have been illustrated and described, it should be understood that changes and modifications can be made therein in accordance with ordinary skill in the art without departing from the invention in its broader aspects as defined in the following claims.

What is claimed is:

1. An artificial synovial fluid, comprising a serum, a chelating agent, and a buffer in an aqueous solution.

2. The artificial synovial fluid of claim 1 wherein the serum comprises bovine calf serum.

3. The artificial synovial fluid of claim 1 further comprising an antibiotic.

4. The artificial synovial fluid of claim 3 wherein the antibiotic comprises sodium azide.

5. The artificial synovial fluid of claim 3 wherein the antibiotic comprises Patricin A.

6. The artificial synovial fluid of claim 1 wherein the chelating agent is chosen from the group comprising Ethylene-Diamine-Tetra-Acetate (EDTA), disodium EDTA, tetra sodium EDTA, and Ethylene Glycol bis (2-Aminoethyl Ether)-N,N,N′,N′-Tetraacetic Acid (EGTA).

7. An artificial synovial fluid, consisting essentially of:
25% to 99.8% bovine calf serum, wherein the bovine calf serum has a protein content of 50 g/l to 60 g/l;
0.01% to 3% Ethylene-Diamine-Tetra-Acetate; and
up to 72.0% deionized water,
wherein the percentages of components are weight to weight of the fluid composition.

8. The artificial synovial fluid of claim 7 wherein the artificial synovial fluid has 33% to 66% serum and 0.01% to 0.74% of EDTA.

9. An artificial synovial fluid, consisting essentially of:
25% to 99.8% bovine calf serum, wherein the bovine calf serum has a protein content of 50 g/l to 60 g/l;
0.1% to 5.0% Sodium Azide;
0.01% to 3% Ethylene-Diamine-Tetra-Acetate; and
up to 72.0% deionized water,
wherein the percentages of components are weight to weight of the fluid composition.

10. The artificial synovial fluid of claim 9 wherein the artificial synovial fluid has 33% to 66% serum and 0.01% to 0.74% of EDTA.

11. An artificial synovial fluid, consisting essentially of:
25% to 99.8% bovine calf serum, wherein the bovine calf serum has a protein content of 50 g/l to 60 g/l;
0.1% to 5.0% Patricin A;
0.01% to 3% Ethylene-Diamine-Tetra-Acetate; and
up to 72.0% deionized water,
wherein the percentages of components are weight to weight of the fluid composition.

12. The artificial synovial fluid of claim 9 wherein the artificial synovial fluid has 33% to 66% serum and 0.01% to 0.74% of EDTA.

13. An artificial synovial fluid, consisting essentially of:
25% to 99.8% bovine calf serum, wherein the bovine calf serum has a protein content of 50 g/l to 60 g/l;
0.1% to 5.0% Patricin A;
0.01% to 3% Ethylene-Diamine-Tetra-Acetate; and
up to 72.0% saline,
wherein the percentages of components are weight to weight of the fluid composition.

14. The artificial synovial fluid of claim 13 wherein the artificial synovial fluid has 33% to 66% serum and 0.01% to 0.74% of EDTA.

15. The artificial synovial fluid of claim 13 wherein the saline is phosphate buffered saline.

16. An artificial synovial fluid, consisting essentially of:
25% to 99.8% bovine calf serum, wherein the bovine calf serum has a protein content of 50 g/l to 60 g/l;
1% to 30% Tris,
0.01% to 3% Ethylene-Diamine-Tetra-Acetate; and
up to 72.0% saline,
wherein the percentages of components are weight to weight of the fluid composition.

17. The artificial synovial fluid of claim 16 wherein the saline is phosphate buffered saline.

18. The artificial synovial fluid of claim 17 wherein the artificial synovial fluid has 33% to 66% serum, 1% to 5% Tris, and 0.01% to 0.74% of EDTA.

19. A method of using the artificial synovial fluid of claim 1 comprising adding the artificial synovial fluid to an implant during an in vitro evaluation of implant performance.

20. The method of claim 19 wherein the implant is a prosthetic joint.

21. The method of claim 19 wherein the evaluation of implant performance is a wear test.

22. A method of making the artificial synovial fluid of claim 1 comprising preheating the serum to 37° C., adding the serum, chelating agent, buffer and aqueous solution according to a desired ratio, mixing the fluid and filtering the fluid.

23. The artificial synovial fluid of claim 1 further comprising an implant.

24. The artificial synovial fluid of claim 23 wherein the implant is a prosthetic joint.

25. A method of using the artificial synovial fluid of claim 7 comprising adding the artificial synovial fluid to an implant during an in vitro evaluation of implant performance.

26. A method of using the artificial synovial fluid of claim 9 comprising adding the artificial synovial fluid to an implant during an in vitro evaluation of implant performance.

27. A method of using the artificial synovial fluid of claim 11 comprising adding the artificial synovial fluid to an implant during an in vitro evaluation of implant performance.

28. A method of using the artificial synovial fluid of claim 13 comprising adding the artificial synovial fluid to an implant during an in vitro evaluation of implant performance.

29. A method of using the artificial synovial fluid of claim 16 comprising adding the artificial synovial fluid to an implant during an in vitro evaluation of implant performance.
